(19) United States
(12) Patent Application Publication
(10) Pub. No.: US 2011/0123476 A1
(43) Pub. Date: May 26, 2011

(54) Adhesive Formulations

(76) Inventors: Mbiya Kapiamba, Cromwell, CT (US); Ahmad Robert Hadba, Middlefield, CT (US)

(21) Appl. No.: 13/012,833

(22) Filed: Jan. 25, 2011

Related U.S. Application Data
(63) Continuation-in-part of application No. 12/124,414, filed on May 21, 2008.
(60) Provisional application No. 60/931,571, filed on May 24, 2007.

(51) Int. Cl. A61K 31/74; C08G 63/06; C08G 73/10; C08G 18/46; A61P 17/02

(52) U.S. Cl. 424/78.06; 528/208; 528/206; 528/321; 528/322; 528/170; 528/78; 528/79; 528/76; 528/80; 528/84; 528/74; 528/75

(57) ABSTRACT

The disclosure relates to biocompatible components useful for forming compositions for use as medical/surgical synthetic adhesives and sealants. Biocompatible components of the present disclosure may include a multifunctional amine or multifunctional polyol core, with isocyanate and/or polyalkylene oxide arms, which may optionally be capped with electrophilic or nucleophilic groups. These biocompatible components may, in embodiments, be combined with optional cross linkers to form adhesive and/or sealant compositions.

### CROSS-REFERENCE TO RELATED APPLICATIONS

This application is a continuation-in-part application of co-pending U.S. patent application Ser. No. 12/124,414, filed on May 21, 2008, which, in turn, claims the benefit of and priority to U.S. Provisional Patent Application No. 60/931,571, filed May 24, 2007, the entire disclosures of each of which are hereby incorporated by reference in their entirety.

### TECHNICAL FIELD

The present disclosure relates to adhesives and sealants formed from synthetic components for medical and surgical use with animal tissues in vivo.

### BACKGROUND OF RELATED ART

In recent years there has developed an increased interest in replacing or augmenting sutures with adhesive bonds. The reasons for this increased interest include: (1) the potential speed with which repair might be accomplished; (2) the ability of a bonding substance to effect complete closure, thus preventing seepage of fluids; and (3) the possibility of forming a bond without excessive deformation of tissue or additional injury to tissue.

Studies in this area, however, have revealed that in order for surgical adhesives to be accepted by surgeons, they should possess various properties. For example, they should exhibit high initial tack and an ability to bond rapidly to living tissue; the strength of the bond should be sufficiently high to cause tissue failure before bond failure; the adhesive should form a bridge, typically a permeable flexible bridge; and the adhesive bridge and/or its metabolic products should not cause local histotoxic or carcinogenic effects.

Several materials useful as tissue adhesives or tissue sealants are currently available. One type of adhesive that is currently available is a cyanoacrylate adhesive. However, there is the possibility that a cyanoacrylate adhesive can degrade to generate undesirable by-products such as formaldehyde. Another disadvantage with cyanoacrylate adhesives is that they can have a high elastic modulus which can limit their usefulness.

Another type of tissue sealant that is currently available utilizes components derived from bovine and/or human sources. For example, fibrin sealants are available. However, as with any natural material, variability in the material is frequently observed and, because the sealant is derived from natural proteins, there may be viral transmission concerns.

It would be desirable to provide a biological adhesive or sealant that is fully synthetic and therefore highly consistent in its properties without the concern of viral transmission. Such a composition should be flexible and biocompatible and should be suitable for use as an adhesive or sealant.

### SUMMARY

The present disclosure provides biocompatible compositions which may be utilized as adhesives, sealants, and the like. In embodiments, the present disclosure provides a biocompatible component including:

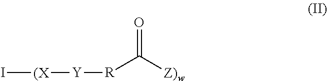

wherein I includes a core derived from a component such as multifunctional polyols and multifunctional polyamines,

X can be carbonate groups, carboxylic acid esters, amides, thioesters, thioamides, urethanes, ureas, thioureas, thiourethanes, and combinations thereof,

Y is derived from a component such as polyalkylene oxides, polyether polyesters, polyether polyurethanes, polyether polyester urethanes, and combinations thereof, bearing terminal hydroxyl or amine groups capable of reacting with X,

Z is derived from a component such as N-hydroxysuccinimide, N-hydroxysulfosuccinimide, pentafluorophenol, p-nitrophenol, and combinations thereof,

R can be alkyl, aryl, ether, and combinations thereof, and

w is a number from about 3 to about 250.

In embodiments, the present disclosure provides a biocompatible composition which includes the above biocompatible component in combination with a cross linker. In such a case, the composition of the present disclosure may include a cross linker; and a biocompatible component including the following formula:

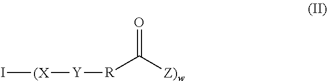

wherein I includes a core derived from a component such as multifunctional polyols and multifunctional polyamines,

X can be carbonate groups, carboxylic acid esters, amides, thioesters, thioamides, urethanes, ureas, thioureas, thiourethanes, and combinations thereof,

Y is derived from a component such as polyalkylene oxides, polyether polyesters, polyether polyurethanes, polyether polyester urethanes, and combinations thereof,

Z is derived from a component such as N-hydroxysuccinimide, N-hydroxysulfosuccinimide, pentafluorophenol, p-nitrophenol, and combinations thereof,

R can be alkyl, aryl, ether, and combinations thereof, and w is a number from about 3 to about 250.

As noted above, the biocompatible composition may, in embodiments, be utilized as an adhesive or sealant.

The present disclosure also provides methods for producing these compositions.

The compositions of the present disclosure can be applied by a variety of methods, including spraying the compositions onto a surgical site. In embodiments, the present disclosure includes methods for closing wounds by applying a composition of the present disclosure to a wound and allowing the composition to set, thereby closing said wound. Such wounds may include, in embodiments, incisions. Compositions of the present disclosure may also be utilized to seal leaks in animals. In embodiments, compositions of the present disclosure may also be utilized to adhere a medical device, such as an implant, to a surface of animal tissue.

### DETAILED DESCRIPTION

The present disclosure relates to biocompatible compositions for use as tissue adhesives or sealants, which are biocompatible, non-immunogenic and biodegradable. The biocompatible compositions can be employed to approximate tissue edges, adhere medical devices (e.g. implants) to tissue, seal air/fluid leaks in tissues, and for tissue augmentation such as sealing or filling voids or defects in tissue. Thus, as used herein, an “adhesive” is understood to include a composition which adheres one thing to another, such as tissue edges to each other or a device, such as an implant, to tissue, and a “sealant” is understood to include a composition which is applied to tissue and utilized to seal air/fluid leaks in tissue or seal or fill small voids or defects in tissue. However, an adhesive composition herein may be used as a sealant, and a sealant composition may be used as an adhesive.

The biocompatible compositions can be applied to living tissue and/or flesh of animals, including humans. While certain distinctions may be drawn between the usage of the terms “flesh” and “tissue” within the scientific community, the terms are used interchangeably herein as referring to a general substrate upon which those skilled in the art would understand the present composition to be utilized within the medical field for the treatment of patients. As used herein, “tissue” may include, but is not limited to, skin, bone, neuron, axon, cartilage, blood vessel, cornea, muscle, fascia, brain, prostate, breast, endometrium, lung, pancreas, small intestine, blood, liver, testes, ovaries, cervix, colon, stomach, esophagus, spleen, lymph node, bone marrow, kidney, peripheral blood, embryonic tissue, and/or ascite tissue.

In accordance with the present disclosure, a biocompatible component is provided which includes a multifunctional core. Suitable cores which may be utilized include, but are not limited to, multifunctional polyols, multifunctional amines, polythiols, and the like. As used herein “multifunctional” includes a core possessing at least 3 functional groups, in embodiments from about 3 to about 250 functional groups, in embodiments from about 4 to about 12 functional groups, in other embodiments from about 5 to about 8 functional groups.

Examples of multifunctional polyols which may be utilized to form a multifunctional core in accordance with the present disclosure include, but are not limited to, polyether polyols; polyester polyols; block copolymers including branched chain ethoxylated alcohols; alkoxylated alcohols such as NEODOL® which is sold commercially by Shell Chemical Company; polyvinyl alcohols; polyhydric alcohols; carboxylic acid esters of polyhydric alcohols; polyglycols; polylactone polyols; combinations thereof, and the like.

In some embodiments, suitable polyols for use as the multifunctional polyol include polyether-based polyols, polyester-based polyols such as polycaprolactone-based polyols, and polyhydric alcohols such as glycerol, pentaerythritol, sorbitol, mannitol, trimethylol propane, diethylene glycol, pentaerythritol ethoxylate, pentaerythritol propoxylate, dipentaerythritiol, combinations thereof, and the like. In some embodiments, the polyol can be glycerol, trimethylol propane, hexane-1,2,6-triol, polycaprolactone triol, or any polyol obtained by partial reaction of any polyol with polyisocyanates, polycarboxylic acid derivatives, combinations thereof, and the like, to create longer polymeric molecules.

Where the multifunctional core is a multifunctional polyol, the polyol can have a molecular weight of from about 130 g/mol to about 20,000 g/mol, in embodiments from about 134 g/mol to about 1000 g/mol.

Examples of multifunctional amines which may be utilized to form a multifunctional core in accordance with the present disclosure include, but are not limited to, poly(allyl amine), poly(L-lysine), polyalkylene oxides having three or more amine functional groups, polyethylene oxide/polypropylene oxide copolymers possessing three or more amine functional groups, trilysine, diethylene triamine, di(heptamethylene) triamine, di(trimethylene) triamine, bis(hexamethylene) triamine, triethylene tetramine, tripropylene tetramine, tetraethylene pentamine, hexamethylene heptamine, pentaethylene hexamine, dimethyl octylamine, dimethyl decylamine, rh-collagen, rh-gelatin, chitosan, combinations thereof, and the like.

Where the multifunctional core is a multifunctional amine, the amine can have a molecular weight of from about 130 g/mol to about 100,000 g/mol, in embodiments from about 132 g/mol to about 10,000 g/mol.

The multifunctional core may, in embodiments, be combined with groups such as polyalkylene oxides (“PAO”), isocyanates, combinations thereof, and the like, which groups may form arms extending from the multifunctional core thereby forming a biocompatible component of the present disclosure.

Suitable polyalkylene oxides which may be combined with a multifunctional core include, but are not limited to, polyethylene glycols (“PEG”), polypropylene glycols (“PPG”), polyethylene oxides (“PEO”), polypropylene oxides (“PPO”), polyethylene glycols with lactide linkages, polypropylene glycol-co-polyethylene oxide block or random copolymers, polyethylene oxide/polypropylene oxide copolymers, sometimes referred to herein as PEO/PPO copolymers or poloxamers, including triblock PEO/PPO copolymers commercially available as PLURONICS® from BASF Corporation (Mt. Olive, N.J.), combinations thereof, and the like.

As noted above, in some embodiments the multifunctional core can be combined with an isocyanate. Suitable isocyanates for combination with the multifunctional core include aromatic, aliphatic and alicyclic isocyanates, including polyisocyanates. Examples include, but are not limited to, aromatic diisocyanates such as 2,4-toluene diisocyanate, 2,6-toluene diisocyanate, 2,2′-diphenylmethane diisocyanate, 2,4′-diphenylmethane diisocyanate, 4,4′-diphenylmethane diisocyanate, diphenyldimethylmethane diisocyanate, dibenzyl diisocyanate, naphthylene diisocyanate, phenylene diisocyanate, xylylene diisocyanate, 4,4′-oxybis(phenyl isocyanate), and/or 2,4,6-trimethyl-1,3-phenylene diisocyanate; aliphatic diisocyanates such as tetramethylxylylene diisocyanate, tetramethylene diisocyanate, hexamethylene diisocyanate, lysine diisocyanate, 2-methylpentane-1,5-diisocyanate, 3-methylpentane-1,5-diisocyanate, hexane-1,6-diisocyanate, and/or 2,2,4-trimethylhexamethylene diisocyanate; and alicyclic diisocyanates such as isophorone diisocyanate, cyclohexane diisocyanate, hydrogenated xylylene diisocyanate, hydrogenated diphenylmethane diisocyanate, and/or hydrogenated trimethylxylylene diisocyanate. In embodiments, combinations of the foregoing isocyanates may be utilized.

In some embodiments, isocyanates which may be combined with the multifunctional cores include, but are not limited to, toluene diisocyanate (TDI), 4,4′-diphenylmethane diisocyanate (MDI), isophorone diisocyanate (IPDI), hexamethylene diisocyanate (HMDI), m-tetramethylxylylene diisocyanate (m-TMXDI), p-tetramethylxylylene diisocyanate (p-TMXDI), and combinations thereof.

In embodiments, the multifunctional core may be combined with multiple groups forming arms thereon, including both isocyanates and polyalkylene oxides noted above. Methods for combining such components are within the purview of those skilled in the art. In embodiments, each free hydroxy group of a multifunctional polyol, or each free amine group of a multifunctional amine, may be combined with an isocyanate, polyalkylene oxide, combinations thereof, and the like.

In embodiments, the multifunctional core may first be combined with a diisocyanate, thereby forming a multifunctional core possessing isocyanate arms. The free isocyanate group of the diisocyanate may then, in embodiments, be reacted with a polyalkylene oxide as described above, thereby forming arms having a diisocyanate adjacent the multifunctional core, followed by a polyalkylene oxide.

In other embodiments, the multifunctional core may first be combined with a polyalkylene oxide, thereby forming a multifunctional core possessing polyalkylene oxide arms. The free hydroxyl groups on the polyalkylene oxide arms may then be reacted, in embodiments, with a diisocyanate as described above. The free isocyanate group of the diisocyanate group may then, in embodiments, be reacted with an additional polyalkylene oxide as described above, thereby forming arms having a polyalkylene oxide adjacent the multifunctional core, followed by a diisocyanate, followed by another polyalkylene oxide.

The free hydroxyl groups of the polyalkylene oxide groups furthest from the multifunctional core may then, in embodiments, be further functionalized with an end group within the purview of those skilled in the art. Examples of such functional groups include nucleophilic groups, electrophilic groups, combinations thereof, and the like.

In some embodiments it may be desirable to functionalize the polyalkylene oxides at the ends of the arms of the biocompatible component of the present disclosure with electrophilic groups. For example, in some embodiments the free hydroxyl groups may be converted to carboxylic groups by reacting them with anhydrides such as succinic anhydride in the presence of tertiary amines such as pyridine or triethylamine or dimethylaminopyridine

(“DMAP”). Other anhydrides which may be utilized include, but are not limited to, glutaric anhydride, phthalic anhydride, maleic anhydride, combinations thereof, and the like. The resultant terminal carboxyl groups may then be converted to an activated ester by reacting with N-hydroxysuccinimide (NHS) and/or N-hydroxysulfosuccinimide (Sulfo-NHS), optionally in the presence of dicyclohexylcarbodiimide (DCC) and/or N-(3-dimethylaminopropyl) carbodiimide (EDC), to produce N-hydroxysuccinimide ester groups, which are electrophilic, at the ends of the arms of the biocompatible component of the present disclosure.

In embodiments, the multifunctional core, the arms, or both, may include degradable linkages so as to render the components of the present disclosure degradable, as well as any composition including these components. Suitable degradable linkages which can be optionally incorporated in the biocompatible component and/or compositions of the present disclosure include, but are not limited to, hydrolytically labile α-hydroxy acids such as lactic acid, glycolic acid, and hydroxy-butyric acid, glycolide, lactide, lactones including ε-caprolactone, carbonates such as trimethylene carbonate, ester ethers such as dioxanones including 1,4-dioxane-2-one and 1,3-dioxane-2-one, diacids including succinnic acid, adipic acid, sebacic acid, malonic acid, glutaric acid, azelaic acid, phosphoesters such as ethyl dichlorophosphate, anhydrides including sebacic acid anhydride and azelaic acid anhydride, combinations thereof, and the like. Those skilled in the art will readily envision reaction schemes for incorporating these degradable linkages into the biocompatible component of the present disclosure.

The biocompatible component of the present disclosure may thus, in embodiments, possess the following formula:

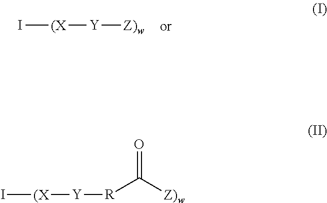

wherein I may be a multifunctional core as described above, for example a multifunctional polyol or a multifunctional polyamine,

X may be derived from a functional group that allows the attachment of a diol or diamine macromer/polymer such as a carboxylic acid, a thiocarbonyl, isocyanate, isothiocyanate, or combinations thereof, so that X is a carbonate, carboxylic acid ester, amide, thioester, thioamide, urethane, urea, thiourea or thiourethane, or combinations thereof, Y may be derived from a polymeric or macromeric diol or diamine, including a polyalkylene oxide, a polyether polyester, a polyether polyurethane, a polyether polyester urethane, or combinations thereof, bearing terminal hydroxyl or amine groups capable of reacting with X,

Z may be derived from a group that forms an activated ester and allows increased reactivity toward amines, such as N-hydroxysuccinimide, N-hydroxysulfosuccinimide, pentafluorophenol, p-nitrophenol, and combinations thereof,

R may be an alkyl, aryl, ether, or combinations thereof, capable of being derived from a reactive diacid or anhydride, and

w may be a number from about 3 to about 250, in embodiments from about 4 to about 12, in other embodiments from about 5 to about 8.

In some embodiments it may be desirable to form an adduct of a diisocyanate with a hydrophilic polymer such as a polyalkylene oxide including ethylene glycol or polyethylene glycol and use the resulting adduct to functionalize a multifunctional core in accordance with the present disclosure. The adduct may be formed by reacting a polyalkylene oxide as described above with a diisocyanate described above, followed by reacting the free cyanate group of the diisocyanate with another polyalkylene oxide.

In yet other embodiments, polyalkylene oxides having functional groups such as succinimidyl groups may be obtained from commercial sources. For example, activated forms of polyethylene glycol described above having electrophilic groups are commercially available from Shearwater Polymers, Huntsville, Ala., and Union Carbide, South Charleston, W. Va. Thus, in some embodiments, these functionalized polyalkylene oxides may be utilized to form arms on a multifunctional core as described above, with no further functionalization necessary.

The resulting adduct may then be added to the multifunctional core so that one of the polyalkylene oxides becomes attached to the multifunctional core while the free polyalkylene oxide is available for activation by the forming of electrophilic groups such as N-hydroxysuccinimide esters as described above. In other embodiments, the adduct may possess one polyalkylene oxide activated with an electrophilic group such as N-hydroxysuccinimide esters as described above, in some embodiments obtained from a commercial source, which may then be attached to the multifunctional core.

The electrophilic groups at the ends of the arms of the biocompatible component of the present disclosure may then be reacted with a nucleophilic group, such as an amine cross linker or a polyol, polythiol or polyphosphine, to produce an adhesive or sealant composition in accordance with the present disclosure. As would be readily apparent to one skilled in the art, the desired properties of the compositions of the present disclosure can be adjusted by the selection of the specific components utilized to prepare the resulting adhesive or sealant compositions.

Suitable amine crosslinkers which may be reacted with the biocompatible component of the present disclosure include those multifunctional amines described above which may be used as the multifunctional core. Amine cross linkers which may be utilized include, for example, primary amines, secondary amines, diamines, aromatic amines, polyamines, polyamidoamines, and combinations thereof. Multifunctional amines may also include primary aliphatic amines, primary aromatic amines, secondary aliphatic or alicyclic amines, and/or secondary aromatic amines. The amine group may be linked to the multifunctional cores by other groups such as ester, amide, ether, amine, combinations thereof, and the like. Suitable amines which may be utilized as the amine cross linker include poly(allyl amine), trilysine, poly(L-lysine), polyalkylene oxides having two or more primary or secondary amine functional groups, spermidine, spermine, 1,4-bis(3-aminopropyl)piperazine, diaminobicyclooctane, and the like.

Other examples of suitable amines which may be used as the at least one amine cross linker include, but are not limited to, triethylamine, diisopropylethylamine, ethylene diamine, 1,4-butane diamine, hexamethylene diamine, isomers of hexamethylene diamine, diethylene triamine, triethylene tetramine, lysine and lysine containing polypeptides, arginine and arginine containing polypeptides, tetraethylene pentamine, bishexamethylene triamine, N,N′-Bis(3-aminopropyl)-1,2-ethane diamine, N-(3-Aminopropyl)-1,3-propane diamine, N-(2-aminoethyl)-1,3 propane diamine, cyclohexane diamine, isomers of cyclohexane diamine, 4,4′-methylene biscyclohexane amine, 4′4′-methylene bis(2-methylcyclohexane amine), isophorone diamine, phenalkylene polyamines, combinations thereof, and the like.

Aromatic amines may also be used as the amine cross linker. Suitable aromatic mines include, for example, di-(4-aminophenyl)sulfone, di-(4-aminophenyl)ether, 2,2-bis(4-aminophenyl)propane, 4,4′-diamino diphenylmethane, 3,3′-dimethyl-4,4′-diaminodiphenyl methane, m-phenylene diamine, p-phenylene diamine, m-xylylene diamine, toluene diamine, 4,4′-methylene dianiline, benzidine, 4,4′-thiodianiline, 4-methoxy-1,3-phenyldiamine, 2,6-diaminopyridine, dianisidine, combinations thereof, and the like.

Polyether diamines may also be utilized as the amine cross linker. Suitable polyether diamines include, but are not limited to, 4,9-dioxadodecane-1,12-diamine, 4,7,10-trioxamidecane-1,12-diamine, bis(3-amino propyl)polytetrahydrofurans, Bis(3-aminopropyl)amine, 1,2-Bis(3-aminopropylamino)ethane, and commercially available polyoxyalkylene amines from Texaco Chemical Co. under the JEFFAMINE® brand as D230, D400, D2000, T403, and T-3000. Combinations of the foregoing polyether diamines may be utilized in embodiments.

In some embodiments, the amine cross linker can be an amino functional polymer such as those sold under the JEFFAMINE® brand, a poly(allyl amine), poly(L-lysine), or other amino functional polymers such as a polyalkylene oxide, including PEG, PEO and PPO having two or more amine functional groups.

Other suitable amine cross linkers include chitosan, recombinant proteins such as rh-collagen, rh-gelatin and rh-albumin, recombinant glycosaminoglycans such as rh-hyaluronic acid, combinations thereof, and the like.

In embodiments, combinations of the foregoing cross linkers may be utilized to form an adhesive composition and/or sealant composition of the present disclosure.

An adhesive composition and/or sealant composition of the present disclosure may thus possess the biocompatible component of the present disclosure in an amount of from about 10 to about 100 percent by weight of the composition, in embodiments from about 50 to about 90 percent by weight of the composition, with the cross linker component of the adhesive composition and/or sealant composition present in an amount of from about 0 to about 90 percent by weight of the composition, in embodiments from about 10 to about 50 percent by weight of the composition.

In some embodiments, the weight ratio of the biocompatible component of the present disclosure to the cross linker in a composition of the present disclosure may be from about 5000:1 to about 2.5:1, in embodiments from about 1000:1 to about 10:1.

The resulting composition of the present disclosure can be used in a medical/surgical capacity in place of, or in combination with, sutures, staples, clamps, combinations thereof, and the like.

Optional components may be added to the composition of the present disclosure to adjust its viscosity according to a specific application of use, e.g., as an adhesive or a sealant. Such optional components can include, for example, diethylene glycol dimethyl ether (“DIGLYME”), dimethylformamide (“DMF”), dimethyl succinate, dimethyl glutarate, dimethyl adipate, combinations thereof, and the like. Thickening agents which can be used to adjust the viscosity of the compositions of the present disclosure include polycyanoacrylates, polylactic acid, polyglycolic acid, lactic-glycolic acid copolymers, poly-3-hydroxybutyric acid, polyorthoesters, polyanhydrides, pectin, combinations thereof, and the like.

Where utilized, such additives can be included so that they are present in an amount of from about 1 to about 30 percent by weight of the composition, in embodiments from about 2 to about 15 percent by weight of the composition.

Optionally, stabilizers can also be added to increase the storage stability of the compositions of the present disclosure. Suitable stabilizers can include those which prevent premature polymerization such as quinones, hydroquinone, hindered phenols, hydroquinone monomethyl ether, catechol, pyrogallol, benzoquinone, 2-hydroxybenzoquinone, p-methoxy phenol, t-butyl catechol, butylated hydroxy anisole, butylated hydroxy toluene, t-butyl hydroquinone, combinations thereof, and the like. Suitable stabilizers can also include anhydrides, silyl esters, sultones (e.g., α-chloro-α-hydroxy-o-toluenesulfonic acid-γ-sultone), sulfur dioxide, sulfuric acid, sulfonic acid, sulfurous acid, lactone, boron trifluoride, organic acids, alkyl sulfate, alkyl sulfite, 3-sulfolene, alkylsulfone, alkyl sulfoxide, mercaptan, alkyl sulfide, combinations thereof, and the like. In some embodiments, an anhydride such as maleic anhydride, sebacic acid anhydride, and/or azelaic acid anhydride, can be used as a stabilizer. In other embodiments antioxidants such as Vitamin E, Vitamin K1, cinnamic acid, and/or flavanone can be used as stabilizers.

Where utilized, such stabilizers can be included so that they are present in an amount from about 0.01 to about 10 percent by weight of the composition, in embodiments from about 0.1 to about 2 percent by weight of the composition.

In some embodiments, solid supported catalysts may be used during synthesis to improve stability of the resulting composition of the present disclosure. The presence of such catalysts may increase reactivity during use. Suitable catalysts are within the purview of those skilled in the art and can include stannous octoate, triethylamine, diethylaminoethanol, dimethylaminopyridine (DMAP), combinations thereof, and the like. The amount of catalyst employed can be from about 0.5 grams to about 50 grams per kilogram of the other components of the composition.

The compositions of the present disclosure can be used for a number of different human and animal medical applications including, but not limited to, wound closure (including surgical incisions and other wounds), adhesives for medical devices (including implants), void fillers, and embolic agents. Adhesive compositions and/or sealant compositions may be used to bind tissue together either as a replacement of, or as a supplement to, sutures, staples, clamps, tapes, bandages, and the like. Use of the disclosed compositions can eliminate or substantially reduce the number of sutures normally required during current practices, and eliminate the subsequent need for removal of staples and certain types of sutures. The compositions of the present disclosure thus can be particularly useful for use with delicate tissues where sutures, clamps or other conventional tissue closure mechanisms may cause further tissue damage.

Application of the compositions of the present disclosure, with or without other additives, can be done by any conventional means. These include dripping, brushing, or other direct manipulation of the composition on the tissue surface, by syringe, such as with a mixer nozzle, or spraying of the composition onto the surface. In open surgery, application by hand, forceps, or the like is contemplated. In endoscopic surgery, the composition can be delivered through the cannula of a trocar, and spread at the site by any device within the purview of those skilled in the art.

In embodiments, the biocompatible component of the present disclosure, optionally in combination with the cross linker, may be dissolved in a solvent to form a solution for application. Suitable solvents include those that are water miscible and biologically acceptable for medical/surgical use. In some embodiments, the solvents can include DIGLYME (diethylene glycol dimethyl ether), N,N-dimethylformamide (“DMF”), dimethyl sulfoxide, combinations thereof, and the like.

In embodiments, the biocompatible component may be in a first solution, with the at least one cross linker dissolved in an aqueous media which optionally contains at least one biodegradable thickener. Suitable biologically acceptable thickeners include disaccharides, polysaccharides, alginates, hyaluronic acid, pectins, dextrans, cellulosics such as carboxymethyl cellulose, methyl cellulose, combinations thereof, and the like.

The biocompatible component may be present in the first solution in an amount from about 10% to about 100% by weight of the first solution, in embodiments from about 50% to about 90% by weight of the first solution. The amount of cross linker in the aqueous media, sometimes referred to herein as a second solution, may be from about 0.01% to about 10% by weight of the second solution, in embodiments from about 0.05% to about 5% by weight of the second solution. Where present, a biodegradable thickener may be present in an amount from about 0% to about 10% by weight of the second solution.

The first component solution and the second cross linker solution may then be combined upon application to form a sealant or adhesive composition of the present disclosure. For example, the composition of the present disclosure can be dispensed from a conventional adhesive dispenser, which may provide mixing of the first and second components prior to the dispenser. Such dispensers are disclosed, for example, in U.S. Pat. Nos. 4,978,336, 4,361,055, 4,979,942, 4,359,049, 4,874,368, 5,368,563, and 6,527,749, the disclosures of each of which are incorporated by reference herein.

In some embodiments, a dual-compartment applicator may be utilized and mixing of the first component solution and second component solution may occur to form an adhesive upon dispensing by an aerosol or by means of a mixing head attached to the applicator or syringe. Other additives can be introduced into the first component solution, the second component solution, or both.

For example, the adhesive composition may be sprayed onto mammalian tissue, which lowers the risk of additional mechanical stress on the tissue. The spray application can be by any means within the purview of those skilled in the art such that the composition can be applied as a fine mist or aerosol. For example, the composition can be placed in a spray bottle and delivered with a hand pump. Alternatively, the composition can be placed in a container with a non-chlorofluorohydrocarbon propellant (e.g., air, nitrogen, carbon dioxide, and/or hydrocarbons) and delivered using a pressurized spray can. In either case, the composition is passed through a fine orifice to form a mist and delivered to the surgical location.

In other embodiments, especially where the composition of the present disclosure is to be utilized as a void filler or to fill a defect in an animal's body, it may be advantageous to more precisely control the conditions and extent of cross-linking; in such a case, it may be desirable to partially cross-link the composition prior to its use to fill a void in animal tissue. The composition of the present disclosure may then be applied to the void or defect and allowed to set, thereby filling the void or defect.

To effectuate the joining of two tissue edges, the two edges may be approximated, and the biocompatible component may be applied in combination with the cross linker. In other embodiments, the biocompatible component may be applied to one tissue edge, the cross linker may be applied to a second tissue edge, and the two edges then brought into contact with each other. The components crosslink rapidly, generally taking less than one minute. The composition of the present disclosure can thus be used as an adhesive to close a wound, including a surgical incision. In such a case, the composition of the present disclosure can be applied to the wound and allowed to set, thereby closing the wound.

In another embodiment, the present disclosure is directed to a method for using the adhesive composition of the present disclosure to adhere a medical device to tissue, rather than secure two edges of tissue. In some aspects, the medical device includes an implant. Other medical devices include, but are not limited to, pacemakers, stents, shunts and the like. In some embodiments, depending on the composition of the medical device, a coating may be required on the medical device. In some aspects such a coating can include the biocompatible component of the present disclosure in combination with the cross linker. Generally, for adhering a device to the surface of animal tissue, the composition of the present disclosure can be applied to the device, the tissue surface, or both. In other embodiments, the biocompatible component of the present disclosure can be applied to either the device or the tissue surface, with the crosslinker applied to the other (i.e., where the biocompatible component has not been applied). The device and tissue surface are then brought into contact with each other and the composition is allowed to set, thereby adhering the device and tissue surface to each other.

The composition of the present disclosure can also be used to prevent post surgical adhesions. In such an application, the composition may be applied and cured as a layer on surfaces of internal tissues in order to prevent the formation of adhesions at a surgical site during the healing process. In addition to the formation of adhesion barriers, in embodiments the adhesive may be utilized to form implants such as gaskets, buttresses or pledgets for implantation.

In another embodiment, the composition can be used to attach skin grafts and position tissue flaps during reconstructive surgery. In still another embodiment, the composition can be used to close tissue flaps in periodontal surgery.

Applications for the compositions of the present disclosure also include sealing tissues to prevent or control blood or other fluid leaks at suture or staple lines. In embodiments, the composition can be used to seal or adhere delicate tissue together in place of conventional tools that may cause mechanical stress. The composition can also be used to seal air and/or fluid leaks in tissue. Additionally, the composition can be applied to tissue as a barrier to prevent adhesions, provide a protective layer for delicate damaged tissue and/or provide a drug delivery layer to a surgical site.

When used as a sealant, the composition of the present disclosure can be used in surgery to prevent or inhibit bleeding or fluid leakage both during and after a surgical procedure. It can also be applied to prevent air leaks associated with pulmonary surgery. The sealant may be applied directly to the desired area in at least an amount necessary to seal off any defect in the tissue and seal off any fluid or air movement.

A variety of optional ingredients including medicinal agents may also be added to the compositions of the present disclosure. These agents may be added to adhesive compositions of the present disclosure, sealant compositions of the present disclosure, or both. A phospholipid surfactant that provides antibacterial stabilizing properties and helps disperse other materials in the compositions may be added to the compositions of the present disclosure. Additional medicinal agents include antimicrobial agents, colorants, preservatives, or medicinal agents such as, for example, protein and peptide preparations, antipyretic, antiphlogistic and analgesic agents, anti-inflammatory agents, vasodilators, antihypertensive and antiarrhythmic agents, hypotensive agents, antitussive agents, antineoplastics, local anesthetics, hormone preparations, antiasthmatic and antiallergic agents, antihistaminics, anticoagulants, antispasmodics, cerebral circulation and metabolism improvers, antidepressant and antianxiety agents, vitamin D preparations, hypoglycemic agents, antiulcer agents, hypnotics, antibiotics, antifungal agents, sedative agents, bronchodilator agents, antiviral agents, dysuric agents, combinations thereof, and the like.

Imaging agents such as iodine, barium sulfate, or fluorine, can also be combined with the compositions of the present disclosure to allow visualization of the surgical area through the use of imaging equipment, including X-ray, MRI, and/or CAT scan.

Additionally, an enzyme may be added to the compositions of the present disclosure to increase their rate of degradation. Suitable enzymes include, for example, peptide hydrolases such as elastase, cathepsin G, cathepsin E, cathepsin B, cathepsin H, cathepsin L, trypsin, pepsin, chymotrypsin, γ-glutamyltransferase (γ-GTP), and the like; sugar chain hydrolases such as phosphorylase, neuraminidase, dextranase, amylase, lysozyme, oligosaccharase, and the like; oligonucleotide hydrolases such as alkaline phosphatase, endoribonuclease, endodeoxyribonuclease, and the like. In some embodiments, where an enzyme is added, the enzyme may be included in a liposome or microsphere to control the rate of its release, thereby controlling the rate of degradation of the composition of the present disclosure. Methods for incorporating enzymes into liposomes and/or microspheres are within the purview of those skilled in the art.

The present compositions have a number of advantageous properties. The resulting compositions of the present disclosure are safe and biocompatible, possess enhanced adherence to tissue, are biodegradable, have hemostatic potential, have low cost, and are easy to prepare and use. The composition has a rapid curing time. Application of the composition, with or without other additives, can be done by any conventional means. By varying the selection of the components, the strength and elasticity of the adhesive and/or sealant composition can be controlled, as can the gelation time.

The compositions rapidly form a compliant gel matrix, which insures stationary positioning of tissue edges or implanted medical devices in the desired location where the composition is utilized as an adhesive, and a tightly adherent yet flexible seal where the composition is used as a sealant. In either case, the rapidity of gelation lowers the overall required surgical/application time. Where delicate or spongy tissues are involved and/or air or fluid leaks must be sealed, spray application of a composition may be utilized to avoid stress to the tissue and insure a uniform coating over the area.

The compositions retain the positional integrity of the tissue to which the composition is applied and/or location of a medical device. The compositions form strong cohesive bonds. They exhibit excellent mechanical performance and strength, while retaining the necessary pliability to adhere living tissue. This strength and pliability allows a degree of movement of tissue without shifting the surgical tissue edge. Additionally, the compositions are biodegradable, allowing the degradation components to pass safely through the subject's body.

It will be understood that various modifications may be made to the embodiments disclosed herein. For example, the compositions in accordance with this disclosure can be blended with other biocompatible, bioabsorbable or non-bioabsorbable materials. As another example, optional ingredients such as dyes, fillers, medicaments or antimicrobial compounds can be added to the composition. Therefore, the above description should not be construed as limiting, but merely as exemplifications of embodiments. Those skilled in the art will envision other modifications within the scope and spirit of the claims appended hereto.

What is claimed is:

1. A biocompatible component comprising the following formula: 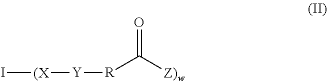
wherein I comprises a core derived from a component selected from the group consisting of multifunctional polyols and multifunctional polyamines,
X is selected from the group consisting of carbonate groups, carboxylic acid esters, amides, thioesters, thioamides, urethanes, ureas, thioureas, thiourethanes, and combinations thereof,
Y is derived from a component selected from the group consisting of polyalkylene oxides, polyether polyesters, polyether polyurethanes, polyether polyester urethanes, and combinations thereof, bearing terminal hydroxyl or amine groups capable of reacting with X,
Z is derived from a component selected from the group consisting of N-hydroxysuccinimide, N-hydroxysulfosuccinimide, pentafluorophenol, p-nitrophenol, and combinations thereof,
R is selected from the group consisting of alkyl, aryl, ether, and combinations thereof, and
w is a number from about 3 to about 250.

2. The biocompatible component of claim 1, wherein the multifunctional polyol is selected from the group consisting of polyether polyols, polyester polyols, branched chain ethoxylated alcohols, alkoxylated alcohols, polyvinyl alcohols, polyhydric alcohols, carboxylic acid esters of polyhydric alcohols, polyglycols, polylactone polyols, and combinations thereof.

3. The biocompatible component of claim 1, wherein the multifunctional polyol is selected from the group consisting of hexane-1,2,6-triol, polycaprolactone triol, glycerol, pentaerythritol, sorbitol, mannitol, trimethylol propane, diethylene glycol, pentaerythritol ethoxylate, pentaerythritol propoxylate, dipentaerythritol, and combinations thereof.

4. The biocompatible component of claim 1, wherein the multifunctional amine is selected from the group consisting of poly(allyl amine), poly(L-lysine), polyalkylene oxides having three or more amine groups, polyethylene oxide/polypropylene oxide copolymers possessing three or more amine groups, trilysine, diethylene triamine, di(heptamethylene) triamine, di(trimethylene) triamine, bis(hexamethylene) triamine, triethylene tetramine, tripropylene tetramine, tetraethylene pentamine, hexamethylene heptamine, pentaethylene hexamine, dimethyl octylamine, dimethyl decylamine, rh-collagen, rh-gelatin, chitosan, and combinations thereof.

5. The biocompatible component of claim 1, wherein the polyalkylene oxide is selected from the group consisting of polyethylene glycols, polypropylene glycols, polyethylene oxides, polypropylene oxides, polyethylene glycols with lactide linkages, polypropylene glycol-co-polyethylene oxide copolymers, polyethylene oxide/polypropylene oxide copolymers, and combinations thereof.

6. The biocompatible component of claim 1, wherein the isocyanate comprises a diisocyanate selected from the group consisting of aromatic diisocyanates, aliphatic diisocyanates and alicyclic diisocyanates.

7. The biocompatible component of claim 6, wherein the diisocyanate is selected from the group consisting of 2,4-toluene diisocyanate, 2,6-toluene diisocyanate, 2,2′-diphenylmethane diisocyanate, 2,4′-diphenylmethane diisocyanate, 4,4′-diphenylmethane diisocyanate, diphenyldimethylmethane diisocyanate, dibenzyl diisocyanate, naphthylene diisocyanate, phenylene diisocyanate, xylylene diisocyanate, 4,4′-oxybis(phenyl isocyanate), 2,4,6-trimethyl-1,3-phenylene diisocyanate, tetramethylxylylene diisocyanate, tetramethylene diisocyanate, hexamethylene diisocyanate, lysine diisocyanate, 2-methylpentane-1,5-diisocyanate, 3-methylpentane-1,5-diisocyanate, hexane-1,6-diisocyanate, 2,2,4-trimethylhexamethylene diisocyanate, isophorone diisocyanate, cyclohexane diisocyanate, hydrogenated xylylene diisocyanate, hydrogenated diphenylmethane diisocyanate, hydrogenated trimethylxylylene diisocyanate, and combinations thereof.

8. A composition comprising:
a cross linker; and
a biocompatible component comprising the following formula: 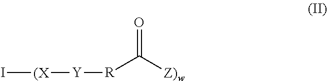
wherein I comprises a core derived from a component selected from the group consisting of multifunctional polyols and multifunctional polyamines,
X is selected from the group consisting of carbonate groups, carboxylic acid esters, amides, thioesters, thioamides, urethanes, ureas, thioureas, thiourethanes, and combinations thereof,
Y is derived from a component selected from the group consisting of polyalkylene oxides, polyether polyesters, polyether polyurethanes, polyether polyester urethanes, and combinations thereof,
Z is derived from a component selected from the group consisting of N-hydroxysuccinimide, N-hydroxysulfosuccinimide, pentafluorophenol, p-nitrophenol, and combinations thereof,
R is selected from the group consisting of alkyl, aryl, ether, and combinations thereof, and
w is a number from about 3 to about 250.

9. The composition of claim 8, wherein the cross linker comprises a polyfunctional amine cross linker selected from the group consisting of primary amines, secondary amines, diamines, aromatic amines, polyamines, polyamidoamines, and combinations thereof.

10. The composition of claim 8, wherein the cross linker comprises an amine cross linker selected from the group consisting of poly(allyl amine), poly(L-lysine), polyalkylene oxides having two or more amine functional groups, spermidine, spermine, 1,4-bis(3-aminopropyl)piperazine, diaminobicyclooctane, triethylamine, diisopropylethylamine, ethylene diamine, 1,4-butane diamine, hexamethylene diamine, diethylene triamine, triethylene tetramine, lysine, lysine containing polypeptides, trilysine, arginine, arginine containing polypeptides, tetraethylene pentamine, bishexamethylene triamine, N,N′-Bis(3-aminopropyl)-1,2-ethane diamine, N-(3-Aminopropyl)-1,3-propane diamine, N-(2-aminoethyl)-1,3 propane diamine, cyclohexane diamine, 4,4′-methylene biscyclohexane amine, 4′4′-methylene bis(2-methylcyclohexane amine), isophorone diamine, phenalkylene polyamines, di-(4-aminophenyl)sulfone, di-(4-aminophenyl)ether, 2,2-bis(4-aminophenyl)propane, 4,4′-diamino diphenylmethane, 3,3′-dimethyl-4,4′-diaminodiphenyl methane, m-phenylene diamine, p-phenylene diamine, m-xylylene diamine, toluene diamine, 4,4′-methylene dianiline, benzidine, 4,4′-thiodianiline, 4-methoxy-1,3-phenyldiamine, 2,6-diaminopyridine, dianisidine, 4,9-dioxadodecane-1,12-diamine, 4,7,10-trioxamidecane-1,12-diamine, bis(3-amino propyl)polytetrahydrofurans, Bis(3-aminopropyl)amine, 1,2-Bis(3-aminopropylamino)ethane, polyoxyalkylene amines, and combinations thereof.

11. The composition of claim 8, wherein the multifunctional polyol is selected from the group consisting of polyether polyols, polyester polyols, branched chain ethoxylated alcohols, alkoxylated alcohols, polyvinyl alcohols, polyhydric alcohols, carboxylic acid esters of polyhydric alcohols, polyglycols, polylactone polyols, and combinations thereof.

12. The composition of claim 8, wherein the multifunctional polyol is selected from the group consisting of hexane-1,2,6-triol, polycaprolactone triol, glycerol, pentaerythritol, sorbitol, mannitol, trimethylol propane, diethylene glycol, pentaerythritol ethoxylate, pentaerythritol propoxylate, dipentaerythritol, and combinations thereof.

13. The composition of claim 8, wherein the multifunctional amine is selected from the group consisting of poly(allyl amine), poly(L-lysine), polyalkylene oxides having three or more amine groups, polyethylene oxide/polypropylene oxide copolymers possessing three or more amine groups, trilysine, diethylene triamine, di(heptamethylene) triamine, di(trimethylene) triamine, bis(hexamethylene) triamine, triethylene tetramine, tripropylene tetramine, tetraethylene pentamine, hexamethylene heptamine, pentaethylene hexamine, dimethyl octylamine, dimethyl decylamine, rh-collagen, rh-gelatin, chitosan, and combinations thereof.

14. The composition of claim 8, wherein the polyalkylene oxide is selected from the group consisting of polyethylene glycols, polypropylene glycols, polyethylene oxides, polypropylene oxides, polyethylene glycols with lactide linkages, polypropylene glycol-co-polyethylene oxide copolymers, polyethylene oxide/polypropylene oxide copolymers, and combinations thereof.

15. The composition of claim 8, wherein the isocyanate comprises a diisocyanate selected from the group consisting of aromatic diisocyanates, aliphatic diisocyanates and alicyclic diisocyanates.

16. The composition of claim 15, wherein the diisocyanate is selected from the group consisting of 2,4-toluene diisocyanate, 2,6-toluene diisocyanate, 2,2′-diphenylmethane diisocyanate, 2,4′-diphenylmethane diisocyanate, 4,4′-diphenylmethane diisocyanate, diphenyldimethylmethane diisocyanate, dibenzyl diisocyanate, naphthylene diisocyanate, phenylene diisocyanate, xylylene diisocyanate, 4,4′-oxybis(phenyl isocyanate), 2,4,6-trimethyl-1,3-phenylene diisocyanate, tetramethylxylylene diisocyanate, tetramethylene diisocyanate, hexamethylene diisocyanate, lysine diisocyanate, 2-methylpentane-1,5-diisocyanate, 3-methylpentane-1,5-diisocyanate, hexane-1,6-diisocyanate, 2,2,4-trimethylhexamethylene diisocyanate, isophorone diisocyanate, cyclohexane diisocyanate, hydrogenated xylylene diisocyanate, hydrogenated diphenylmethane diisocyanate, hydrogenated trimethylxylylene diisocyanate, and combinations thereof.

17. The composition of claim 8, wherein the biocompatible component of claim 1 is present in an amount from about 50 to about 90 percent by weight of the composition, and the cross linker is present in an amount from about 10 to about 50 percent by weight of the composition.

18. A method for closing a wound comprising:
applying the composition of claim 8 to said wound; and
allowing the composition to set thereby closing said wound.

19. A method for sealing a leak in animal tissue comprising:
applying the composition of claim 8 to said leak; and
allowing the composition to set thereby sealing said leak.

20. A method for adhering a medical device to a surface of animal tissue comprising:
applying the composition of claim 8 to said device, said surface or both;
bringing the device, composition and surface into contact with each other; and
allowing the composition to set thereby adhering the device and surface to each other.
